(19) United States
(12) Patent Application Publication
(10) Pub. No.: US 2007/0123844 A1
(43) Pub. Date: May 31, 2007

(54) Method for treating ocular disorders

(76) Inventor: Shelly Henry, Yukon, OK (US)

Correspondence Address: DUNLAP, CODDING & ROGERS P.C., PO BOX 16370, OKLAHOMA CITY, OK, 73113, US

(21) Appl. No.: 11/582,789

(22) Filed: Oct. 18, 2006

Related U.S. Application Data
(60) Provisional application No. 60/728,233, filed on Oct. 19, 2005.

(51) Int. Cl. A61B 18/18; A61N 5/06

(52) U.S. Cl. 606/4; 607/88

(57) ABSTRACT

The present invention is an ophthalmological treatment process utilizing a low energy (“low power”, “low level”) laser applied through the sclera of the eye to treat various disease conditions of the eye, including but not limited to, blindness. This process is used to create the optimum environment for healing by providing energy to the ocular tissues causing these ocular tissues, in particular the retina, to have improved function regarding, for example, light sensitivity and transmission.

### CROSS-REFERENCE TO RELATED APPLICATIONS

The present application claims the benefit under 35 U.S.C. 119(e) of U.S. Provisional Application 60/728,233 filed Oct. 19, 2005, the entire contents of which is hereby expressly incorporated herein by reference.

### BACKGROUND

Many heritable and non-heritable disease conditions of the eye involve complete loss or severe impairment of vision blindness. Many of these ocular disease conditions involve destruction of all or portions of the retina, the light-sensitive inner portion of the eye which transforms light energy to electrical energy for stimulation of the optic nerve. Partial blindness due to diseases of the retina has heretofore been very difficult to treat. Usually, even if the disease condition can be arrested, the vision loss which has already occurred prior to treatment is permanent.

One treatment of retinal diseases is known as photodynamic therapy (PDT), as described, for example, in U.S. Pat. Nos. 6,162,242; 6,622,729; 6,800,086; 6,942,655; and 7,060,695 (each of which is hereby expressly incorporated herein by reference in its entirety).

PDT is used to selectively target a desired area of the body (in this case, the eye) for treatment. A photosensitive agent or drug is administered, then after a certain time period a light source with a wavelength corresponding to the absorbance spectrum of the administered photosensitive agent is targeted to the particular site for treatment. A laser is preferably used to direct the light to only the specific tissue or area to be treated. The time period between administration of the agent and phototreatment is usually between about 1-60 minutes. The photosensitive agent, upon activation by the defined wavelength of light, produces cytotoxic oxygen radicals. These radicals disrupt microvascular structures in the treatment area and result in subsequent tissue damage.

However, PDT has disadvantages in that it must be used with pretreatment with a photosensitizing agent, it acts by causing damage to microvascular tissues, and it does not restore nor necessarily improve the subject's vision (though it may stop further degeneration of the vision).

Novel treatments for blindness are therefore desperately needed which do not cause further tissue damage and which can help to restore lost vision as well as inhibit further vision loss. It is to this object that the present invention is directed.

### SUMMARY

The present invention is an ophthalmological treatment process utilizing a low energy (“low power”, “low level”) laser applied through the sclera of the eye to treat various disease conditions of the eye, including but not limited to, blindness. This process is used to create the optimum environment for healing by providing energy to the ocular tissues causing these ocular tissues, in particular the retina, to have improved function regarding, for example, light sensitivity and transmission.

### BRIEF DESCRIPTION OF THE DRAWINGS

FIG. 1 is a diagram of an eye having various laser application points indicated thereon.

FIG. 2 is a diagram of an eye wherein the eyeball has been moved downwardly to increase exposure an area of the sclera above the cornea.

FIG. 3 is a diagram of an eye wherein the eyeball has been moved upwardly to increase exposure an area of the sclera below the cornea.

FIG. 4 is a diagram of an eye wherein the eyeball has been moved to the right to increase exposure an area of the sclera to the left of the cornea.

FIG. 5 is a diagram of an eye wherein the eyeball has been moved to the left to increase exposure of an area of the sclera to the right of the cornea.

FIG. 6 is a cross sectional view through an eyeball showing a laser in an application position near the eye.

### DETAILED DESCRIPTION OF THE INVENTION

The present invention is an ophthalmological treatment process utilizing a low energy (“low power”, “low level”) laser applied through the sclera of the eye to treat various disease conditions of the eye, including but not limited to, blindness. The present invention contemplates a type of low energy, non-heating, non-cutting photodynamic therapy wherein no photosensitive agent is used in the treatment, contrary to other methods of PDT known in the art. The present invention is therefore a non-photosensitive agent-based photodynamic therapy (non-PSA PDT) and does not involve light energy levels sufficient to cut or heat tissues. This process is used to create the optimum environment for healing by providing energy to the ocular tissues to causing these ocular tissues, in particular the retina, to have improved function regarding, for example, light sensitivity and transmission.

The present invention relies on utilizing particular points and zones on the eye, wherein the light from the laser is thereby directed at differing angles directly into the eye, preferably avoiding transmission through the cornea. Using the properties of light to carry the energy through refraction, reflection, diffraction and wave properties, the light energy is delivered to the inner surface of the eye to portions thereof such as the retina.

Among the vision disorders that can be treated using the methods contemplated herein are severe visual impairment (i.e., blindness), including diseases related to degeneration of cells of the retina and macula, including Usher syndrome, Stargardt disease, Bardet-Biedl syndrome, Best disease, choroideremia, gyrate-atrophy, retinitis pigmentosa, macular degeneration, Leber Congenital Amaurosis (Leber's Hereditary Optic Neuropathy), Blue-cone monochromacy, retinoschisis, Malattia Leventinese, Oguchi Disease, and Refsum disease, or other diseases related to impairment of the function of the retina or macula.

Other macular degeneration disorders may include but are not limited to any of a number of conditions in which the retinal macula degenerates or becomes dysfunctional, e.g., as a consequence of decreased growth of cells of the macula, increased death or rearrangement of the cells of the macula (e.g., RPE cells), loss of normal biological function, or a combination of these events such as North Carolina macular dystrophy, Sorsby's fundus dystrophy, pattern dystrophy, dominant drusen, and any condition which alters or damages the integrity or function of the macula (e.g., damage to the RPE or Bruch's membrane). For example, the term macular degeneration encompasses retinal detachment, chorioretinal degenerations, retinal degenerations, photoreceptor degenerations, RPE degenerations, mucopolysaccharidoses, rod-cone dystrophies, cone-rod dystrophies and cone degenerations.

The light source used in the present invention is a low level laser commonly known in the art as a “cold laser” or “low power laser”. The light wavelengths used in the present invention may range from 200 nm to 2000 nm and preferably are in the wavelength range from 600-1000 nm, more preferably from 650-850 nm, even more preferably from 25-830 nm, and most preferably from 775 to 815 nm. The laser may be used at energy levels (mW/cm²) from 1 m W to 100 mW, to 1 W, to 2 W, to 3 W, to 4 W, to 5 W, to 6 W, to 7 W, to 8 W, to 9 W, and to 10 W. More particularly the energy level of the laser may be from 5 mW to 800 mW, from 50 mW to 750 mW, from 100 mW to 700 mW, from 200 mW to 650 mW, from 250 mW to 600 mW, from 300 mW to 550 mW, from 350 mW to 550 mW, from 350 mW to 550 mW, from 400 mW to 550 mW, from 450 mW to 550 mW, from 475 mW to 525 mW, and most preferably at 490-510 mW.

In one embodiment, the non-heating low level laser (cold laser) used is an Acumed laser commercially available from Laser Thera Corp. Preferably the laser is used at a power level of from 20 mW to 500 mW, but may be any non-cutting, non-heating power level commonly known and used in the art as a “cold light” or “low level” laser. Other low level lasers that may be used include but are not limited to the MicroLight 830 (Microlight Corp. Of Am. Missouri City, Tex.), the Axiom Biolaser, LLLT Series 3 (Axiom Worldwide, Tampa Fla.), the Acculaser Pro4 (Photothera, Carlsbad, Calif.), the Thor DDII IR lamp system (Thor Intl. Ltd, Amersham, U/S), the Thor DDII 830 CL3 laser system, and the Thor 810 nm IR, 450 mW, and Thor 810 nm IR, 200 mW.

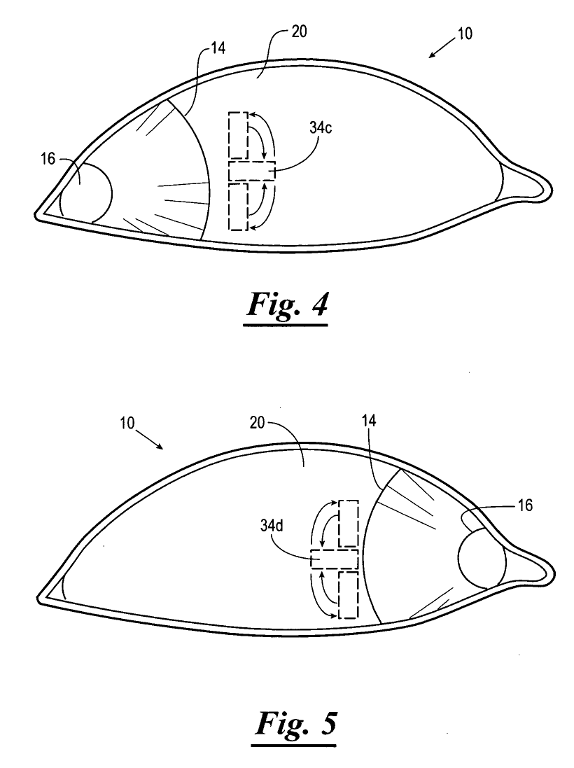

In alternate embodiments (shown for example in U.S. Ser. No. 60/728,233 previously incorporated by reference herein), the laser device may be modified to have one or more light emitting portions which rotate in a circular orientation about a central point (see FIG. 3 therein). As further shown in FIG. 3 therein, the laser device may comprise a plurality of concentrically located diodes (light emitting portions) which rotate about a central point, in the same or opposite direction. Alternatively the laser device my comprise a plurality of concentrically-oriented diodes which are stationary (see FIG. 4 therein).

Without wishing to be bound by theory, the beneficial physiological results of the low level laser device used in the present invention may include: an increase in absorption of light and nutrients, a decrease in intraocular pressure, an increase in drainage, an increase in muscular strength in the eye, an increase in vascular circulation in the eye, an increase in vision, a decrease in scar tissue, acceleration of regeneration of nerve tissue, reattachment of detached tissue, repairs of nervous tissue, an increase in tissue repair, an increase in endorphins, an increase in ATP production, an increase in nerve conduction latency, an increase in mast cells, an increase in neutrophilis, an increase in fibroblasts, an increase in macrophage activity, an increase in endothelial cells, a decrease in inflammation, an increase in perfusion and oxygeneration, an increase in satellite cells, a decrease in healing time, an increase in metabolic activity, an increase in beneficial enzymes, a decrease in deleterious enzymes, an increase in ATP and NO in the presence of LELT which enhances cellular metabolism, stimulation of Cytochrome c Oxydase (a chromophore found in the mitochondria of cells) which causes the rapid increase in ATP synthesis, improved perfusion, an increase in the transport of nutrients and oxygen to the damaged cells and facilitation of repair and removal of cellular debris, increased leukocytic activity which results in enhanced removal of non-viable cellular and tissue components allowing rapid repair and regeneration, improved angiogenesis, increased nitric oxide production to enhance nerve cell perfusion and oxygenation, improvement in nerve latency values, induced axonal sprouting, increased release of acetylcholine that normalizes nerve signal transmission, ion channel normalization, and increased fibroblast proliferation.

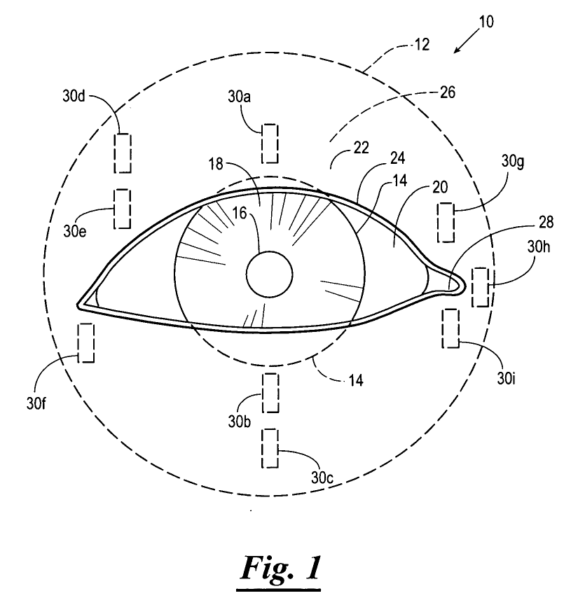

Turning now to the figures, shown in FIG. 1 is a diagram of a human eye 10. The human eye 10 has an eyeball 12 in an eye socket (not shown). The eyeball 12 further comprises a cornea 14, a pupil 16, an iris 18, a sclera 20 (which forms the “white” outer wall of the eyeball 12), and an eye lid 22 having an eye lid edge 24 disposed about a frontal portion of the eyeball 12. The eye lid 22 conceals a covered portion 26 of the sclera 20. A tear duct 28 is shown at a corner of the eye lid 22.

Indicated on the frontal surface of the eye lid 22 are a plurality of laser application positions 30a-30i. These laser application positions 30a-30i approximate the locations of standard acupuncture positions on the eyeball 12. The laser application positions 30a-30i are located above the sclera 20 of the eyeball 12. Each laser application position 30a-30i is preferably at least 2 mm from the eye lid edge 24. The spatial area covered by each laser application position 30a-30i is approximately the same spatial area covered by the beam from the laser, for example about 1 cm×3 mm. In a preferred embodiment the laser application position 30a is approximately directly above the pupil 16 and the laser application positions 30b and 30c are approximately directly below the pupil 16. Laser application positions 30f and 30i are approximately directly below the right and left corners of the eye 10, respectively. Positions 30d and 30e are approximately above and slightly to the left of the right corner of the eye 10, and position 30g is approximately above and slightly to the right of the left corner of eye 10. Position 30h is preferably approximately to the left of the left corner of the eye 10.

During a preferred course of treatment a beam from a low level laser (cold laser) as described elsewhere herein is applied to at least one laser application position 30a-30i (or more preferably all positions 30a-30i) for approximately 10 seconds each. Each application of the laser beam to each position is hereinafter referred to as a “laser application”. Each set of “laser applications” (e.g., one set of laser applications to one or more of laser application positions 30a-30i is referred to hereinafter as a “laser treatment”. A set of laser treatments is referred to herein as “treatment series” (e.g., three laser treatments in a single day is a “treatment series”). The number of treatment series which is conducted over a period of days or weeks is referred to herein as a “treatment protocol”. Treatment protocols are determined on a case by case basis depending on the severity, type and cause of visual impairment of the patient, although treatment protocols generally last from one to nine weeks, or longer, then on a follow-up basis at a rate, for example of one series of treatments per week for maintenance. A laser treatment in which the laser applications are applied at laser application positions 30a-30i (i.e., through the eye lid 22) is referred to herein as a “Type 1” laser application.

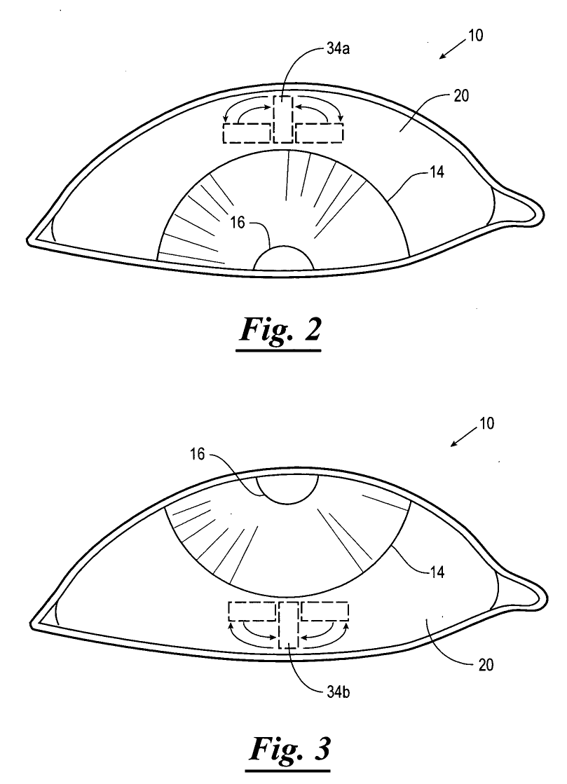

Laser applications may also be performed directly through the sclera 20, as shown in FIGS. 2-5. A laser treatment conducted in this manner directly through the sclera 20 is referred to herein as a “Type 2” laser treatment. In this embodiment of the invention, the laser application occurs at at least one of positions 34a-d (and preferably all of 34a-d) at positions above, below, to the left (the patient's left) of and to the right (the patient's right) of the patient's cornea 14, respectively, wherein the laser beam is applied directly through the sclera 20.

In a Type 2 treatment, the approximately rectangular face of the laser beam is swept across the sclera 20 through an angle of from about 60° to about 180° (as shown), for about 5 seconds in each laser application. As shown in FIGS. 2-5, the patient moves the eyeball 12 downwardly (FIG. 2), upwardly (FIG. 3), to the right (FIG. 4) and to the left (FIG. 5), to expose the sclera 20. The laser application is then applied as indicated at positions 34a-d, respectively (though not necessarily in that order), wherein each set of four (or fewer) laser applications is referred to as a Type 2 laser treatment. A set of Type 2 laser treatments (e.g., three) is referred to as a Type 2 treatment series, and the total number of Type 2 treatment series which is conducted over a period of time is referred to as a treatment protocol.

A treatment protocol may be comprised of both Type 1 and Type 2 treatments or only Type 1 or Type 2 treatments, or other treatment types not described herein. In one embodiment of a Type 1 or Type 2 treatment protocol, a treatment series may comprise one laser treatment, or two, three, or more laser treatments, each separated by a rest period of from 1 to 30 minutes, for example, with an optimal rest period of 5-15 minutes, and more preferably 8-10-12 minutes. For a Type 1 or Type 2 treatment, each laser application may last for 2-30 seconds, (for example), or more preferably from 5-20 seconds, or 10-15 seconds. For a Type 1 treatment, each laser application preferably is about 10 seconds and for a Type 2 treatment each laser application preferably is about 5 seconds when the power setting on the laser is 500 mW. The durations of the laser applications may be increased beyond the durations listed herein (or even decreased if desired) if a laser is used at a wattage level below 500 mW.

For a Type 1 and/or Type 2 treatment protocol, the patient may be given 5 treatment sets over 5 days in the first week, for example, over 5 consecutive days (or non-consecutive days). In week two, the patient may be given 4 treatment sets over 4 days (e.g., over four consecutive or non-consecutive days). In weeks 3-6, the patient may receive three treatment sets per week over 3 consecutive or non-consecutive days and in weeks 7-9, the patient may receive one or two treatment sets per week. Further treatment may occur in weeks 10 and thereafter, if so determined by the health provider. It will be understood that each treatment set may comprise fewer or more than the laser applications shown herein for each Type 1 or Type 2 treatment set. Further, each treatment protocol may comprise fewer than or more than the number of treatment sets and treatment series described in the embodiments herein.

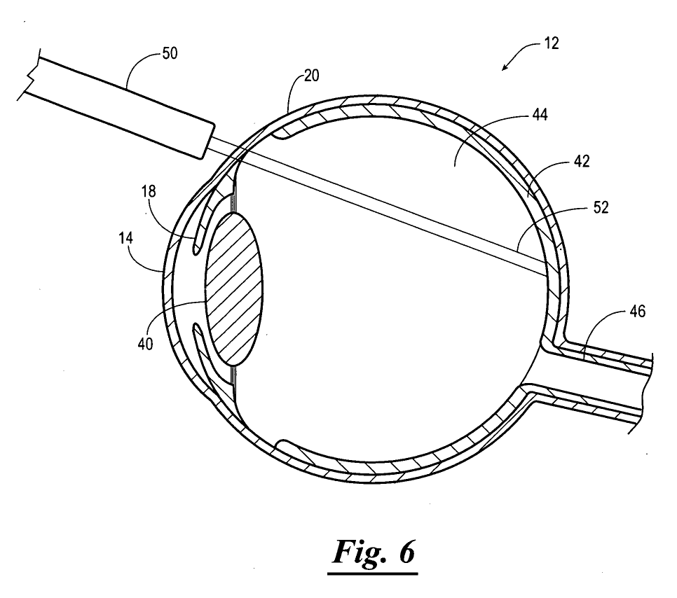

In an exemplary version of a laser application contemplated herein, as shown in FIG. 6, a laser 50 is positioned adjacent an eyeball 12 of a patient, and a laser beam 52 from the laser 50 is directed through the sclera 20, into the vitreous humour 44 and onto a portion of the retina 42 where it causes stimulation of cells of the retina, macula or choroid portions of the eyeball 12. The laser beam 52 generally is directed into the eyeball at an angular orientation of 0 to 90° to the surface of the eyeball 12.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined in the description herein and by the appended claims. Thus, the following examples will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLES

### Example 1

Patient with Leber's disease. The test subject was a 27 year old male who had lost central vision in both eyes at the age of 21. After the first cold laser treatment he could see outlines but still did not have unobstructed vision. After the sixth treatment he could see at 20/200 in his right eye and 20/300 in his left. The treatments were carried out daily for one week. Individual treatments comprised 10-20 laser diode pulses over 3-5 minutes in positions on the eye as described elsewhere herein. Also treated were acupuncture points to decrease signs of depression and anxiety. The laser used in this study was a 808 nm variable laser (Acumed) with settings from 200 mW to 500 mW. The energy setting of the cold laser used in this case was 500 mW.

### Example 2

Patient with Retinitis Pigmentosa. Dustin (21 year old male) was diagnosed with retinitis pigmentosa degenerative eye disease in both eyes at age 5. Upon examination he was legally blind 20/400 left and 20/350 right. He had lost 40% of his peripheral vision in both eyes. Treatment was started fourteen treatments were administered over a one month period. After the treatment series Dustin could then see 20/50 left and 20/60 right, gaining 100% of his peripheral vision.

### Example 3

Patient with Retinitis Pigmentosa. Katie (26 year old female) was diagnosed with retinitis pigmentosa degenerative eye disease in both eyes and 20/400 right, count fingers at 6 inches in left. She had 15 treatments over one month and after the treatment series was seeing 20/100 in both eyes, thereby going from 60% obstruction of vision to 20% obstruction of vision in both eyes.

Changes may be made in the formulation of the various compositions described herein or in the steps or the sequence of steps of the methods described herein without departing from the spirit and scope of the invention as described and claimed herein.

What is claimed is:

1. A method of treating a patient having impaired vision, comprising:
providing a low energy laser;
positioning the low energy laser adjacent the sclera of an eyeball of the patient; and
directing a plurality of pulses of light from the low energy laser through the sclera into the eyeball wherein the pulses of light are applied to the retina of the eyeball in a plurality of locations, and wherein the method is carried out without the addition of a photosensitive agent to the eyeball or the vessels of the eyeball.

2. The method of claim 1 wherein the impaired vision of the patient is due to retinal and macular degeneration, Usher syndrome, Stargardt disease, Bardet-Biedl syndrome, Best disease, choroideremia, gyrate-atrophy, retinitis pigmentosa, Leber Congenital Amaurosis (Leber's Hereditary Optic Neuropathy), Blue-cone monochromacy, retinoschisis, Malattia Leventinese, Oguchi Disease, and Refsum disease, retinal detachment, chorioretinal degenerations, retinal degenerations, photoreceptor degenerations, RPE degenerations, mucopolysaccharidoses, rod-cone dystrophies, cone-rod dystrophies, cone degenerations, conditions involving decreased growth of cells of the macula, increased death or rearrangement of the RPE cells of the macula, North Carolina macular dystrophy, Sorsby's fundus dystrophy, pattern dystrophy, dominant drusen, and any condition which alters or damages the integrity or function of the macula.

3. The method of claim 1 wherein the light wavelengths used in the laser are in the range from 200 nm to 2000 nm.

4. The method of claim 3 wherein the light wavelengths used in the laser are in the range from 600 nm to 900 nm.

5. The method of claim 1 wherein the low energy laser is used at an energy level of 1 mW/cm² to 10 W/cm².

6. The method of claim 5 wherein the low energy laser is used at an energy level of 20 mW/cm² to 750 mW/cm².
